(19) United States
(12) Patent Application Publication
(10) Pub. No.: US 2023/0165704 A1
(43) Pub. Date: Jun. 1, 2023

(54) HYOLARYNGEAL SUSPENSION FOR TREATING SLEEP DISORDERED BREATHING

(71) Applicant: Vanderbilt University, Nashville, TN (US)

(72) Inventor: David T. KENT, Nashville, TN (US)

(21) Appl. No.: 17/920,710

(22) PCT Filed: Apr. 21, 2021
(86) PCT No.: PCT/US2021/028418

§ 371 Date: Oct. 21, 2022

Related U.S. Application Data
(60) Provisional application No. 63/013,678, filed on Apr. 22, 2020.

(51) Int. Cl. A61F 5/56; A61N 1/36

(52) U.S. Cl. CPC A61F 5/56; A61N 1/3611

(57) ABSTRACT

Hyolaryngeal suspension devices and systems for treating sleep disordered breathing (SDB) are provided. A device includes a first fastener sized and configured to anchor to a hyoid bone or thyroid cartilage of the patient and a second fastener is sized and configured to anchor to the sternum, clavicle, or ribs of the patient. A static or elastic member has one end connected to the first fastener and an opposing end connected to the second fastener. The elastic member has sufficient elasticity to allow the pharynx and/or the larynx to elevate dynamically to permit speech and swallowing. The static member has sufficient tension to maintain appropriate haryngeal wall tension while still permitting sufficient movement of the hyoid bone and thyroid cartilage for speech and swallowing function.

### CROSS-REFERENCE TO RELATED APPLICATIONS

The present application claims priority to U.S. Provisional Application No. 63/013,678 filed on Apr. 22, 2020. The contents of which are incorporated by reference in its entirety.

### TECHNICAL FIELD

The present disclosure relates to systems and devices for hyolaryngeal suspension to treat sleep disordered breathing.

### BACKGROUND

Sleep disordered breathing (SDB) occurs when there is a partial or complete cessation of breathing that occurs many times throughout the night. Obstructive sleep apnea (OSA) is a type of SDB that involves cessation or significant decrease in airflow in the presence of breathing effort. It is the most common type of SDB and is characterized by recurrent episodes of upper airway collapse daring sleep inducing repetitive pauses in breathing followed by reductions in blood oxygen saturation or neurologic arousal. The pathophysiology of OSA. can involve factors such as craniofacial anatomy, airway collapsibility, and neuromuscular control of the upper airway dilator musculature. Electromyogram studies have shown that the tonic and phasic activity of the pharyngeal airway dilatory muscles (such as the genioglossus muscle) is progressively reduced from wakefulness to non-rapid eye movement to rapid eye movement.

Continuous positive airway pressure (CPAP) therapy is the frontline treatment for OSA. CPAP therapy utilizes machines, generally including a flow generator, tubing, and a mask designed to deliver a constant flow of air pressure to keep the airways continuously open in patients with OSA. However, the success of CPAP therapy is limited by compliance with reported rates ranging from 50% to 70%. Hypoglossal nerve stimulation (HNS) has now been established as an effective form of therapy for patients with obstructive sleep apnea (OSA) who are unable to tolerate positive airway pressure. This therapy works by protruding and stiffening the tongue muscle thereby dilating the pharyngeal airway. However, only a small subset of patients with OSA have anatomy suitable for hypoglossal nerve stimulation therapy, as many patients continue to suffer from airway collapse even with stimulation of hypoglossal nerve musculature.

There are currently systems approved for statically anchoring the hyoid bone to the back of the jaw but such systems provide static suspension and can cause problems with swallowing, for example, because they restrict the hyoid bone and the rest of the larynx from elevating superiorly and posteriorly to protect the airway from transiting foot boluses and liquid. These systems dilate the airway by placing anterior traction on the hyoid bone, reducing collapse of the base of the tongue and providing a localized stiffening effect on the pharyngeal soft tissues at the level of the tongue base to additionally reduce collapse. They are less effective at stiffening the airway at the level of the oropharynx or velopharynx.

### SUMMARY

Hyolaryngeal suspension devices and systems for treating sleep disordered breathing

(SDB) are provided. In an embodiment, a first fastener is sized and configured to anchor to a hyoid bone or thyroid cartilage of the patient and a second fastener is sized and configured to anchor to the sternum, clavicle, or ribs of the patient. A static or elastic member has one end connected to the first fastener and an opposing end connected to the second fastener. The elastic member has sufficient elasticity to allow the pharynx and/or the larynx to elevate dynamically to permit speech and swallowing. The static member has sufficient tension to maintain appropriate pharyngeal wall tension while still permitting sufficient movement of hyoid bone and thyroid cartilage for speech and swallowing function

### BRIEF DESCRIPTION OF THE DRAWINGS

FIG. 1 is a schematic illustration of a system of the present disclosure implanted in a patient according to an embodiment of the present disclosure.

FIG. 2 is a block diagram of a neuromodulation system according to an embodiment of the present disclosure.

FIG. 3 is a block diagram of a neuromodulation system according to an embodiment of the present disclosure.

### DETAILED DESCRIPTION

As used herein with respect to a described element, the terms “a,” “an,” and “the” include at least one or more of the described element including combinations thereof unless otherwise indicated. Further, the terms “or” and “and” refer to “and/or” and combinations thereof unless otherwise indicated. It will be understood that when an element is referred to as being “over,” “on,” “attached” to, “connected” to, “coupled” with, “contacting,” “in communication with,” etc., another element, it can be directly over, on, attached to, connected to, coupled with, contacting, or in communication with the other element or intervening elements may also be present. In contrast, when an element is referred to as being “directly over,” “directly on,” “directly attached” to, “directly connected” to, “directly coupled” with, “directly contacting,” or in “direct communication” with another element, there are no intervening elements present. An element that is disposed “adjacent” another element may have portions that overlap or underlie the adjacent element. By “substantially” is meant that the shape, configuration, or orientation of the element need not have the mathematically exact described shape, configuration or orientation but can have a shape, configuration or orientation that is recognizable by one skilled in the art as generally or approximately having the described shape, configuration, or orientation. The terms “inferior” and “superior” refer to the position of a human being in standard anatomical position. The systems and devices disclosed herein are used for medical purposes and therefore its components are sterile. As used herein, a “patient” includes a mammal such as a human being.

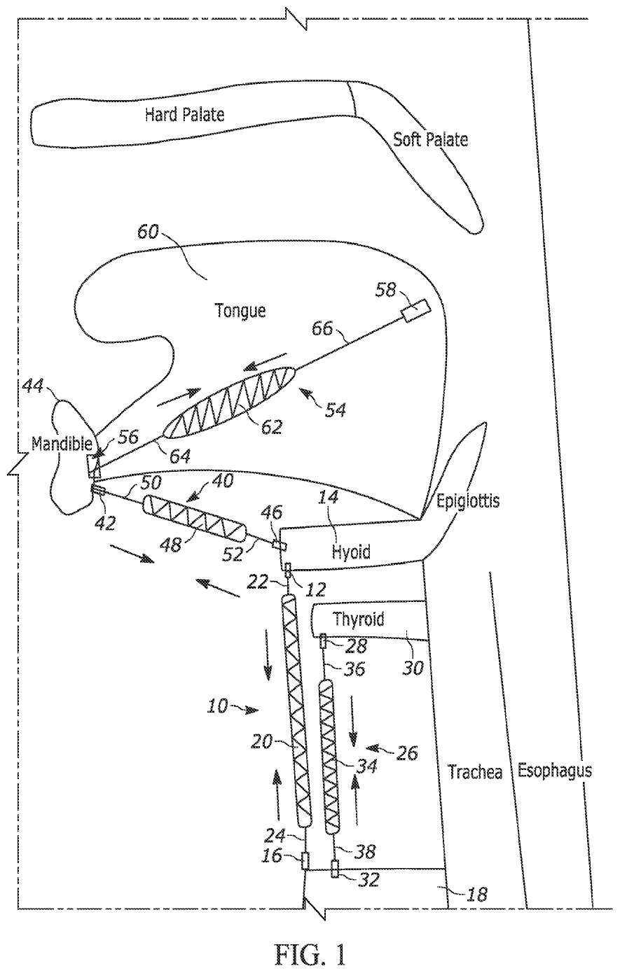

Systems and devices for providing inferior traction to maintain vertical tension in the airway to resist airway collapse are provided. Referring to FIG. 1, in an embodiment, a hyolaryngeal suspension device 10 for treating SDB can comprise first fastener 12 sized and configured to anchor to hyoid bone 14 of a patient and second fastener 16 sized and configured to anchor to the sternum, clavicle, or ribs 18 of the patient. Device 10 can further comprise static or elastic member 20 having one end 22 connected to first fastener 12 and opposing end 24 connected to second fastener 16. The fastener could be, for example, a single fastener, multiple fasteners including miniplates, bone or soft tissue screws, suture loops in combination or alone. In addition or alternatively, a hyolaryngeal suspension device 26 can include first fastener 28 sized and configured to anchor to a thyroid cartilage 30 of a patient and second fastener 32 sized and configured to anchor to the clavicle 18, sternum, or ribs of the patient. Device 26 can further comprise static or elastic member 34 having one end 36 connected to first fastener 28 and opposing end 38 connected to second fastener 32. In addition or alternatively, a hyolaryngeal suspension device 40 can comprise first fastener 42 sized and configured to anchor to mandible 44, such as the back of the mandible for example, of a patient and second fastener 46 sized and configured to anchor to hyoid bone 14 of the patient. Device 40 can further include static or elastic member 48 having one end 50 connected to first fastener 42 and an opposing end 52 connected to second fastener 46, In addition or alternatively, a hyolaryngeal suspension device 54 can include first fastener 56 sized and configured to anchor to mandible 44 of a patient and second fastener 58 sized and configured to anchor to tongue 60 of the patient. Device 54 can further comprise static or elastic member 62 having one end 64 connected to first fastener 56 and opposing end 66 connected to second fastener 58. Systems are also provided that can include one or more of devices 10, 26, 40, and 54.

Any of the embodiments can include a second separate elastic member attached to the respective bones to avoid twisting about a single axis. When attached to the hyoid bone or the thyroid cartilage, the elastic member can have sufficient elasticity to allow the hyoid bone or thyroid cartilage, respectively, to move inferiorly and allow the larynx to elevate dynamically to permit speech and swallowing. By tethering the hyoid bone and/or thyroid cartilage inferiorly, the device can maintain or improve vertical tension in the airway between the larynx and the clavicle, for example. The elastic member can maintain a moderate degree of tension at night, that could be titratable at the time of implant or afterwards, to keep the pharynx from becoming too compliant at night. In certain embodiments, devices are connected directly to the hyoid bone or thyroid cartilages to directly tension the pharynx, and would not do so by proxy through tracheal tension. When attached to the tongue, the elastic member can minimize posterior displacement of the tongue during sleep. The elastic member can be, for example, a spring or filament.

In certain embodiments, a static member attached between the fasteners can restrict hyoid bone and/or thyroid cartilage movement above a certain length away from an inferior fastener. A static member can be titratable at the time of implant or afterwards to keep the pharynx from becoming too compliant at night while still permitting adequate movement for speech and swallowing functions.

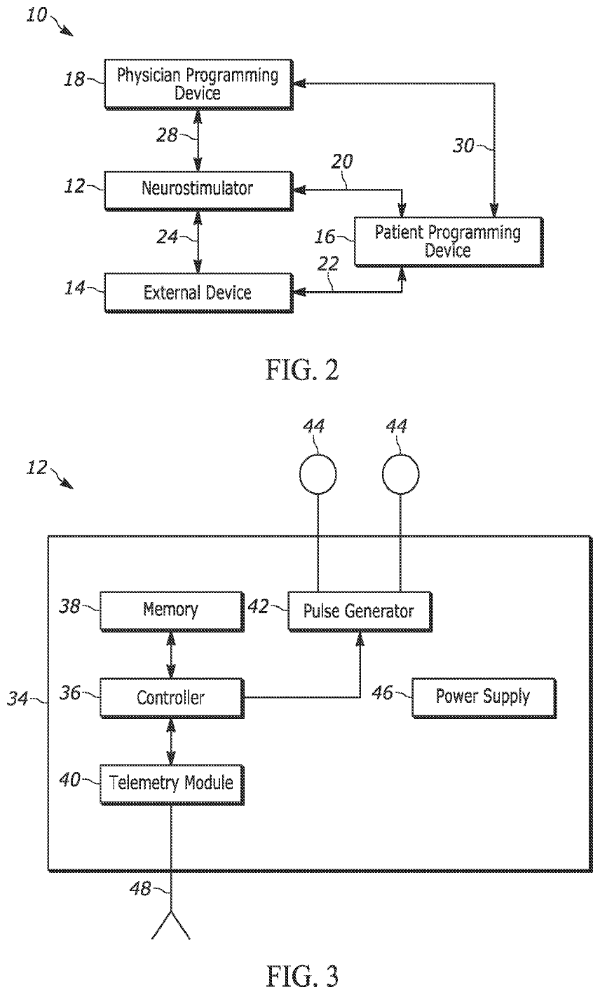

Systems and devices as used herein can be used in addition to positive airway pressure devices, oral appliances, nerve stimulators, such as hypoglossal nerve stimulators, and/or muscle stimulators to treat SDB. For example, a system could further include a neuromodulation system comprising at least one electrode configured to deliver an electrical signal to a target site comprising an upper airway muscle or a nerve innervating an upper airway muscle to activate an upper airway muscle; a power source in electrical communication with the electrode; and a controller in electrical communication with the electrode and programmed to direct delivery of the electrical signal to the target site to stimulate the upper airway muscle or the nerve innervating the upper airway muscle to activate the upper airway muscle to improve the SDB as schematically depicted in FIGS. 2 and 3.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance.

What is claimed is:

1. A hyolaryngeal suspension system to improve sleep disordered breathing (SDB) in a patient comprising:
a thyroid cartilage fastener sized and configured to anchor to a thyroid cartilage of the patient;
an inferior bone fastener sized and configured to anchor to an anatomical bony structure inferior to the thyroid cartilage of the patient; and
an elastic or static member disposed between the thyroid cartilage fastener and the inferior bone fastener, the elastic member having sufficient elasticity to allow the pharynx and/or the larynx to elevate to permit speech and swallowing by the patient, and the static member having sufficient tension to maintain pharyngeal wall tension while still allowing movement of the hyoid bone and thyroid cartilage to permit speech and swallowing by the patient.

2. The hyolaryngeal suspension system of claim 1, wherein the inferior bone fastener is sized and configured to anchor to a sternum, a rib, or a clavicle.

3. The hyolaryngeal suspension system of claim 1, wherein thyroid cartilage traction device provides isolated and sole inferior traction of the thyroid cartilage.

4. The hyolaryngeal suspension system of claim 1, further comprising:
a neuromodulation system comprising:
at least one electrode configured to deliver an electrical signal to a target site comprising an upper airway muscle or a nerve innervating an upper airway muscle to activate an upper airway muscle;
a power source in electrical communication with the electrode; and
a controller in electrical communication with the electrode and programmed to direct delivery of the electrical signal to the target site to stimulate the upper airway muscle or the nerve innervating the upper airway muscle to activate the upper airway muscle to improve the SDB.

5. The hyolaryngeal suspension system of claim 4, wherein the nerve is the ansa cervicalis and the muscle is the sternothyroid muscle.

6. The hyolaryngeal suspension system of claim 1, further comprising:
a hyoid bone fastener sized and configured to anchor to a hyoid bone of the patient; and
an elastic or static member disposed between the thyroid cartilage fastener and the hyoid bone fastener, the elastic member having sufficient elasticity to allow the pharynx and/or the larynx to elevate to permit speech and swallowing by the patient, and the static member having sufficient tension to maintain pharyngeal wall tension while still allowing movement of the hyoid bone and thyroid cartilage to permit speech and swallowing by the patient.

7. The hyolaryngeal suspension system 1, further comprising:
a hyoid bone fastener sized and configured to anchor to a hyoid bone of the patient; and
an elastic or static member disposed between the hyoid fastener and an inferior bone fastener sized and configured to anchor to an anatomical bony structure inferior to the hyoid bone, the elastic member having sufficient elasticity to allow the pharynx and/or the larynx to elevate to permit speech and swallowing by the patient, and the static member having sufficient tension to maintain pharyngeal wall tension while still allowing movement of the hyoid bone and thyroid cartilage to permit speech and swallowing by the patient.

8. The hyolaryngeal suspension system of claim 4, wherein the inferior bone fastener sized and configured to anchor to an anatomical bony structure inferior to the hyoid bone is sized and configured to anchor to a sternum, a rib, or a clavicle.

9. The hyolaryngeal suspension system of claim 4, wherein the inferior bone fastener sized and configured to anchor to an anatomical bony structure inferior to the hyoid bone is the same inferior bone fastener of claim 1 that is sized to anchor to an anatomical bony structure inferior to the thyroid cartilage of the patient.

10. The hyolaryngeal suspension system of claim 1, further comprising:
a mandibular fastener sized and configured to anchor to a mandible of the patient;
a hyoid bone fastener sized and configured to anchor to a hyoid bone of the patient; and
a static or elastic member disposed between the mandibular fastener and the hyoid bone fastener, the elastic member having sufficient elasticity to allow the pharynx and/or the larynx to elevate to permit speech and swallowing by the patient, and the static member having sufficient tension to maintain pharyngeal wall tension while still allowing movement of the hyoid bone and thyroid cartilage to permit speech and swallowing by the patient. 11 The hyolaryngeal suspension device of claim 1, further comprising:
a mandibular fastener sized and configured to anchor to a mandible of a patient;
a tongue fastener sized and configured to anchor to a tongue of the patient; and
an elastic member disposed between the mandibular fastener and the tongue fastener, the elastic member having sufficient elasticity to allow the tongue to move to permit speech and swallowing.

12. The hyolaryngeal suspension device of claim 1, further comprising:
a hyoid bone fastener sized and configured to anchor to a hyoid bone of the patient; and
an elastic or static member disposed between the hyoid bone fastener and the thyroid cartilage fastener or disposed between the hyoid bone fastener and the inferior bone fastener;
a mandibular fastener sized and configured to anchor to a mandible of the patient; and
an elastic or static member disposed between the mandibular fastener and the hyoid bone fastener;
a tongue fastener sized and configured to anchor to a tongue of the patient; and an elastic or static member disposed between the mandibular fastener and the tongue fastener, wherein all of the elastic members have sufficient elasticity to allow the pharynx and/or the larynx to elevate to permit speech and swallowing by the patient, and wherein all of the static members having sufficient tension to maintain pharyngeal wall tension while still permitting sufficient movement of the hyoid bone and thyroid cartilage to permit speech and swallowing by the patient.

13. A hyolaryngeal suspension system to improve sleep disordered breathing (SDB) in a patient comprising:
a hyoid bone mechanical caudal traction system comprising:
a hyoid bone fastener sized and configured to anchor to a hyoid bone of the patient;
an inferior bone fastener sized and configured to anchor to an anatomical bony structure inferior to the hyoid bone of the patient; and
an elastic or static member disposed between the hyoid bone fastener and the inferior bony structure fastener, the elastic member having sufficient elasticity to allow the pharynx and/or the larynx to elevate to permit speech and swallowing by the patient, and the static member having sufficient tension to maintain pharyngeal wall tension while still allowing movement of the hyoid bone and thyroid cartilage to permit speech and swallowing by the patient, wherein the hyoid bone traction system provides isolated and sole inferior traction of the hyoid bone.

12. A hyolaryngeal suspension system to improve sleep disordered breathing (SDB) in a patient comprising:
a hyoid bone mechanical traction system comprising:
a hyoid bone fastener sized and configured to anchor to a hyoid bone of the patient;
an inferior bone fastener sized and configured to anchor to an anatomical bony structure inferior to the hyoid bone of the patient; and
an elastic or static member disposed between the hyoid bone fastener and the inferior bony structure fastener, the elastic member having sufficient elasticity to allow the pharynx and/or the larynx to elevate dynamically to permit speech and swallowing, and the static member having sufficient tension to maintain appropriate pharyngeal wall tension while still allowing sufficient movement of the hyoid bone and thyroid cartilage for speech and swallowing function; and
a neuromodulation system comprising:
at least one electrode configured to deliver an electrical signal to a target site comprising an upper airway muscle or a nerve innervating an upper airway muscle to activate an upper airway muscle;
a power source in electrical communication with the electrode; and
a controller in electrical communication with the electrode and programmed to direct delivery of the electrical signal to the target site to stimulate the upper airway muscle or the nerve innervating the upper airway muscle to activate the upper airway muscle to improve the SDB.

13. The hyolaryngeal suspension system of claim 11, wherein the nerve is the hypoglossal nerve and the muscle is the genioglossus muscle.

14. The hyolaryngeal suspension system of claim 11, wherein the nerve is the glossopharyngeal nerve.

15. The hyolaryngeal suspension system of claim 11, wherein the nerve is the ansa cervicalis and the muscle is the sternothyroid muscle.
